(19) United States
(12) Patent Application Publication
(10) Pub. No.: US 2025/0228523 A1
(43) Pub. Date: Jul. 17, 2025

(54) SYSTEMS AND METHODS FOR IMPROVED IMAGING WITH INTRAVASCULAR ULTRASOUND (IVUS)

(71) Applicants: Case Western Reserve University, Cleveland, OH (US); THE CLEVELAND CLINIC FOUNDATION, Cleveland, OH (US); The United States Government as Represented by The Department of Veteran Affairs, Washington, DC (US)

(72) Inventors: Steve Majerus, University Hts., OH (US); Aaron Fleischman, Beachwood, OH (US)

(21) Appl. No.: 19/016,291

(22) Filed: Jan. 10, 2025

Related U.S. Application Data
(60) Provisional application No. 63/720,239, filed on Nov. 14, 2024.
(60) Provisional application No. 63/663,432, filed on Jun. 24, 2024.
(60) Provisional application No. 63/620,468, filed on Jan. 12, 2024.

(51) Int. Cl. A61B 8/12; G01S 7/52

(52) U.S. Cl. CPC A61B 8/12; G01S 7/52096

(57) ABSTRACT

Systems and methods can facilitate wide broad-band IVUS for peripheral vascular applications with simultaneous improved resolution and penetration depth. A custom analog front end interface can withstand large voltages and recover to receive a sub-mV pulse from less than one mm away. The custom analog front end interface can be on an interface chip that can be sized to be integrated within a tip of the IVUS catheter to connect micro-cables within the IVUS catheter to the broad-band transducer. The interface chip can also include a dual-stage amplifier having a pulse mode configured to withstand voltage pulses greater than 30 V_{PP} during pulsing and an echo reception mode configured to receive sub-mV echoes during echo reception.

### CROSS-REFERENCE TO RELATED APPLICATIONS

This application claims the benefit of U.S. Provisional Application No. 63/720,239, filed Nov. 14, 2024, entitled “SYSTEMS AND METHODS FOR IMPROVED IMAGING WITH INTRAVASCULAR ULTRASOUND (IVUS)”, as well as U.S. Provisional Application No. 63/663,432, filed Jun. 24, 2024, entitled “RAPID RECOVERY, PULSE TOLERANT LOW-NOISE AMPLIFIER FOR BROADBAND ULTRASOUNDS IMAGING”, and U.S. Provisional Application No. 63/620,468, filed Jan. 12, 2024, entitled “RAPID RECOVERY, PULSE TOLERANT LOW-NOISE AMPLIFIER FOR BROADBAND ULTRASOUNDS IMAGING”. These provisional applications are hereby incorporated by reference in their entirety for all purposes.

### GOVERNMENT FUNDING

This invention was made with government support under HL156154 awarded by the National Institutes of Health. The government has certain rights in the invention.

### TECHNICAL FIELD

The present disclosure relates generally to intravascular ultrasound (IVUS) and more particularly to systems and methods that employ a dual-stage amplifier to improve both resolution and penetration depth for IVUS imaging.

### BACKGROUND

An intravascular ultrasound (IVUS) can be used to record real-time images of interior structures of a person's vasculature at a penetration depth and a spatial resolution, enabling real-time investigation during a procedure. Generally, IVUS employs a specially designed, thin catheter with a one or more miniaturized ultrasound transducer attached to the distal end of the catheter. The one or more ultrasonic transducers can be polymer-based (e.g., one or more capacitive micromachined ultrasonic transducers (CMUTs) or one or more piezoelectric micromachined ultrasonic transducers (PMUTs)) and on an interface chip embedded at the tip of the catheter. CMUTs are often used for multi-channel transducer arrays and typically operate at frequencies less than 10 MHz. PMUTs have lower cost and can operate at higher frequencies than CMUTs and generally use high-sensitivity, low-impedance crystalline materials, like lead-zirconium titanate (PZT) or polymers.

Generally, transducers with a higher frequency have a high resolution (good image quality) but poor penetration depth, while transducers with lower frequency have a lower resolution (poorer image quality), but deeper penetration depth. IVUS has been used routinely during interventions within the coronary vasculature that does not require deeper penetration depth. Recently, IVUS has been recommended for use within the peripheral vasculature which requires both high resolution and deeper penetration depth. However, smaller catheters are necessary for use within the peripheral vasculature and the ultrasonic transducers need to be further scaled down to fit leading to problems achieving the necessary high resolution and deeper penetration depth. Current IVUS systems cannot meet this need, trading off either better penetration depth or resolution, and the costs of further miniaturization are exorbitant.

### SUMMARY

Described herein are systems and methods that employ a dual-stage amplifier to facilitate intravascular ultrasound (IVUS) imaging with simultaneous improved resolution and penetration depths. Notably, the dual-stage amplifier allows an IVUS system to achieve improved penetration depth and microscopic resolution simultaneously without an exorbitant cost.

In an aspect, the present disclosure can include a system comprising an intravascular ultrasound (IVUS) catheter, an ultrasound transducer (e.g., a polymeric ultrasound transducer, but may be of one or more different materials), and an interface chip. As an example, the IVUS catheter can have a diameter of 1 mm or less (however, other diameters are possible). The ultrasound transducer can have a broad excitation bandwidth. The interface chip can be sized to be integrated within a tip of the IVUS catheter to connect micro-cables within the IVUS catheter to the ultrasound transducer. The interface chip can also include a dual-stage amplifier having a pulse mode configured to withstand voltage pulses on a level of volts (e.g., greater than 30 V_{PP}) during pulsing and an echo reception mode configured to receive sub-mV echoes during echo reception.

In another aspect, the present disclosure can include a dual-stage amplifier that can connect an ultrasound transducer (e.g., a polymeric ultrasound transducer, but may be of one or more different materials) of an IVUS with a controller. The dual-stage amplifier can include a transimpedance amplifier, an active limited comprising a pulsing channel and an echo reception channel, and a buffer. The pulsing channel can be configured to withstand voltage pulses on a level of volts (e.g., greater than 30 V_{PP}) and the echo reception channel can be configured to receive sub millivolt (mV) echoes. The dual-stage amplifier can fit within a tip of an IVUS catheter (e.g., having a diameter of 1 mm or less; but in some instances the diameter may be larger).

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIGS. 1-2 are block diagrams showing a system for intravascular ultrasound (IVUS);

FIG. 3 shows illustrations of example implementations of the system of FIGS. 1 and 2;

FIG. 4 is a block diagram of the dual-stage amplifier of FIGS. 1 and 2;

FIG. 5 is a block diagram of an example implementation of the dual-stage amplifier of FIG. 4;

FIG. 6 is a process flow diagram illustrating a method for intravascular ultrasound of an artery or vein with the system of FIGS. 1 and 2;

FIG. 7 is a process flow diagram illustrating a method of use of the interface chip of the system of FIGS. 1 and 2;

FIG. 8 is a pictorial example of an interface chip showing a size comparison;

FIG. 9 shows example electrical diagrams of the shunt and the series duplexer circuits;

FIG. 10 shows example electrical diagrams for the interface chip with two channels;

FIG. 11 shows pictorial examples of the interface chip on a test circuit;

FIGS. 12-14 are graphical representations of results of using the interface chip for IVUS;

FIG. 15 shows pictorial examples of a testing setup and an IVUS image of a phantom;

FIG. 16 is a graphical representation of results from testing setup of FIG. 15.

### DETAILED DESCRIPTION

### I. Definitions

Unless otherwise defined, all technical terms used herein have the same meaning as commonly understood by one of ordinary skill in the art to which the present disclosure pertains.

As used herein, the singular forms “a,” “an,” and “the” can also include the plural forms unless the context clearly indicates otherwise.

As used herein, the terms “comprises” and/or “comprising,” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “intravascular ultrasound” or “IVUS” refers to a medical imaging methodology specifically using a specially designed catheter with one or more ultrasound transducer attached to the distal end of the catheter. In some instances, the catheter and one or more ultrasound transducer can be miniaturized to fit within at least a portion of the peripheral vasculature.

As used herein, the term “catheter” refers to a flexible tube-like object that is inserted into a body cavity through a narrow opening. As an example, the catheter can enter into the body's vasculature to at least facilitate entry of a medical devices (e.g., a transducer) for imaging purposes.

As used herein, the term “ultrasound” refers to an imaging method that produces images (also referred to as sonograms) from sound waves interacting with structures of a patient's body.

As used herein, the term “transducer” refers to a device that can convert one form of energy to another.

As used herein, the term “ultrasound transducer” refers to a device that produces sound waves that bounce off body tissues to create echoes and records the echoes. A computing device with a memory and processor, linked to a display, can be attached to the ultrasound transducer to receive the recorded echoes and at least create and/or display an image based on the echoes. In some instances, the ultrasound transducer can be a polymeric ultrasound transducer that can have a broad excitation bandwidth (e.g., 1 mV-300 V, 0.5 mV-175 V, 0 V-110 V, etc.).

As used herein, the term “resolution” refers to “spatial resolution” reflective of clarity of an imaged produced via an ultrasound. Spatial resolution is dependent on axial and lateral resolution, both of which depend on the frequency of the ultrasound.

As used herein, the term “penetration depth” refers to the depth to which sound beams are transmitted and received.

As used herein, the term “multistage amplifier” refers to an electronic device including two or more single stage amplifiers connected together to increase the amplitude of multiple electrical signals. A multistage amplifier can include multiple transistors or transistor pairs.

As used herein, the term “application-specific integrated circuit” or “ASIC” refers to an integrated circuit (IC) chip customized for a particular use, rather than intended for a general-purpose use.

As used herein, the term “patient” refers to any warm-blooded organism including, but not limited to, a human being, a pig, a rat, a mouse, a dog, a car, a goat, a sheep, a horse, a monkey, an ape, a rabbit, a cow, etc., undergoing an IVUS procedure.

### II. Overview

Intravascular ultrasound (IVUS) can be used to record real-time images of interior structures of a patient's vasculature at a high spatial resolution, enabling real-time investigation during a procedure. Recently, IVUS has been recommended for use within the peripheral vasculature, which would require smaller catheters and ultrasonic transducers that are scaled down to fit at the tip of the smaller catheter. At the same time, IVUS requires the ultrasonic transducers to provide an improved penetration depth and as well as high spatial resolution at a microscopic scale. However, like all imaging techniques, IVUS has tradeoffs in imaging depth, resolution, and cost, among other factors. High-frequency IVUS (>40 MHz) has high axial and lateral resolution, but reduced penetration depth, and significant blood speckle which reduces image contrast. Low frequency IVUS trades off resolution for penetration depth. Some approaches use combination transducers operating at different frequencies to achieve both high resolution and penetration depth with a single IVUS catheter, but such approaches are often prohibitively expensive.

The systems and methods described herein can provide both improved resolution and improved penetration depth needed to use IVUS within peripheral vasculature at a lower cost. The IVUS systems can include broad-band polymer transducers. As an example, the broad-band polymer transistor can be a piezoelectric poly[(vinylidenefluoride-co-trifluoroethylene]transducer (PVDF-TrFE) PMUTs) in a sub-mm catheter, which can be attached to a controlling computer with micro-cables via a custom analog front end (AFE) application specific integrated circuit (ASIC) with a dual-stage amplifier. The AFE ASIC can safely withstand pulses greater than at least a voltage (in some instances 30 V_{PP}, 50 V_{PP}, 60 V_{PP}, 70 V_{PP}, 80 V_{PP}, 90 V_{PP}, 100 V_{PP} or higher) while recovering quickly enough to receive sub-mV echoes from targets that are fairly close to the transducer (e.g., located 1 mm, 0.9 mm, 0.8 mm, 0.7 mm, 0.6 mm, 0.5 mm or less from the transducer). The ASIC dimensions can be less than 0.5 mm in width to enable flip-chip integration at the tip of an IVUS catheter (e.g., less than 3 French (1 mm diameter) to fit within a catheter capable of fitting within the smaller peripheral arteries and veins).

### III. Systems

Intravascular ultrasound (IVUS) imaging is commonly used to examine the interior structures of blood vessels in order to study atherosclerotic lesions and/or for coronary interventions including stent placement. IVUS offers penetration depths of up to 10 mm through multiple layers of plaque, vascular tissue, and stent structures and the non-linear reflections allow spectroscopic analysis of plaque composition. However, IVUS imaging has been limited to larger (e.g., more central) veins and arteries by the size of the IVUS system (e.g., the catheter, transducer(s), etc.), the required spatial resolution for useful imaging, and miniaturized component costs. The devices and systems described herein employ a dual-stage amplifier to facilitate IVUS imaging with simultaneous resolution and penetration depths. In some instances, the dual-stage amplifier can be configured for use within veins and/or arteries having an interior diameter of 2 mm or smaller, 1.5 mm or smaller, 1 mm or smaller, 0.5 mm or smaller, or the like (e.g. peripheral veins and/or arteries).

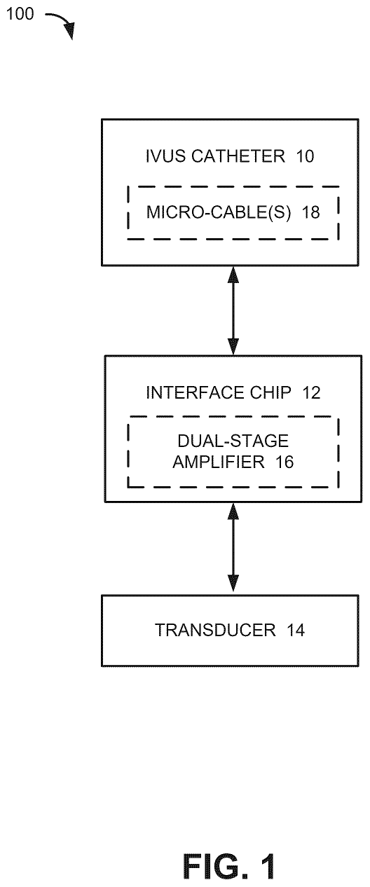

FIG. 1 shows a system 100 for IVUS imaging that can include an IVUS catheter 10, an interface chip 12, and a transducer 14. The IVUS catheter 10 can be a tube (having one or more lumen) of a length having a first end and a second end with at least one lumen extending at least partially there between. The tube can be flexible. The first end can be the tip proximal to the imaging target and the second end can be distal from the imaging target. As an example, the IVUS catheter 10 can have a diameter of 2 mm or smaller a diameter of 1.5 mm or smaller, a diameter of 1 mm or smaller, a diameter of 0.5 mm or smaller, or the like (however, the diameter may be larger limited by the size of the required IVUS catheter). The IVUS catheter 10 can have any length, diameter, material composition, components, and/or other characteristics known in the field of vascular catheters. However, it should be understood that the IVUS catheter may be, or may be combined with, another type of medical device that can provide a mechanism to hold micro-cables 18 and at least part of an interface chip 12 and/or a transducer 14 for imaging purposes. The IVUS catheter 10 can be moved in up to 6 degrees of freedom, for instance the IVUS catheter can be translated forwards and/or backwards through an artery and/or vein, can be rotated (e.g., 360°, 270°, 180°, or the like), can be pitched and/or yawed, or the like to move through the vasculature. It should be understood that more or less degrees of freedom may be available. A guidewire, piezoresistive mechanism, or other motion mechanisms may be used to move the IVUS catheter through the vasculature.

The interface chip 12 can be an application-specific integrated circuit (ASIC) positioned within a tip (e.g., the first end) of the IVUS catheter 10 and can provide an electrical connection between the transducer 14 and micro-cables 18 within the IVUS catheter 10. The micro-cables 18 can provide a connection between the interface chip 12 and a power source and/or controller (neither shown in FIG. 1) outside the IVUS catheter 10. For example, the interface chip 12 can have a custom analog front end (AFE) application specific integrated circuit (ASIC) with a dual-stage amplifier 18 embedded thereon. The AFE ASIC can safely withstand pulses on the level of volts (in some instances, 30 V_{PP}, 50 V_{PP}, 60 V_{PP}, 70 V_{PP}, 80 V_{PP}, 90 V_{PP}, 100 V_{PP} or higher) while recovering quickly enough to receive sub-mV echoes from targets that are fairly close to the transducer 14 (e.g., located 1 mm, 0.9 mm, 0.8 mm, 0.7 mm, 0.6 mm, 0.5 mm or less from the transducer). The ASIC dimensions can be less than 0.5 mm in width to enable flip-chip integration at the tip of an IVUS catheter 10.

The transducer 14 can be an ultrasound transducer having a broad excitation bandwidth. As an example, the ultrasound transducer can be a polymeric ultrasound transducer. The polymeric ultrasound transducer (e.g., transducer 14) can be, for instance, a piezoelectric poly[(vinylidenefluoride-co-trifluoroethylene](PVDF-TrFE) piezoelectric micromachined ultrasonic transducer (PMUT). While shown and described as a single transducer 14 for ease of illustration and explanation it should be understood that one or more transducers 14 of any shape, size, and or configuration capable of appropriate imaging can be used. Alternatively, and/or additionally, a capacitive micromachined ultrasonic transducer (CMUT) may be utilized as part of transducer 14. CMUTs are often for multi-channel transducer arrays, while PMUTs have lower cost and can operate at higher frequencies. CMUTs typically operate lower than 10 MHz, but PMUTs have higher operation frequency (such as high-frequency IVUS having frequencies of 40 MHz or greater) and generally use high-sensitivity, low-impedance crystalline materials like lead-zirconium titanate (PZT). In some instances, a combination of CMUT and PMUT based transducers operating at different frequencies can be used to achieve both high resolution and penetration depth with a single IVUS catheter.

FIG. 2 shows a system 110 similar to 100—having all the elements of system 100 but showing additional components and detail. The transducer 14 can be at least partially attached to, embedded in, positioned on, and/or integrated into the tip (e.g., the first end) of the IVUS catheter 10 to emit ultrasound pulses towards imaging targets (e.g., image target). It is noted that the IVUS catheter 10 is shown with cut ends (e.g., wavy sides) to indicate that the catheter may encompass more or less of the transducer 14, interface 12, and/or micro-cables 18, depending on configuration. FIG. 2 shows the IVUS catheter 10 entirely encompassing the interface 12 and at least partially including the micro-cable(s) 18 and the transducer 14, but this should be understood to be only one example configuration for ease of illustration.

The interior of the IVUS catheter 10 can include the at least one micro-cable 18 that can connect at least the controller 20 and/or the power source 28 to the interface chip 12. The at least one micro-cables 18 can be, for instance, within at least one lumen of the IVUS catheter 10, built into a wall of the tube body of the IVUS catheter, or the like. The interface chip 12 (e.g., the AFE-ASIC) can be sized to be integrated within a tip (the first end) of the IVUS catheter 10 (e.g., can have a width less than 1 mm, and preferably a width less than 0.5 mm, to enable flip chip integration into the catheter tip). The interface chip 12 can connect the at least one micro-cable 18 within the IVUS catheter 10 to the ultrasound transducer (e.g., transducer 14). The interface chip 12 can include a dual-stage amplifier 16 that can have a pulsing mode and an echo reception mode. For example, the pulsing mode can withstand voltage pulses greater than 10 V_{PP}, 20 V_{PP}, 30 V_{PP}, etc. during pulsing (e.g., emission of ultrasound pulses) and the echo reception mode can receive sub-mV echoes during echo reception. The dual-stage amplifier 16 can amplify the pulsing signal and recover quickly enough (e.g., at a rate of 1000 ns or less, 750 ns or less, 500 ns or less, 300 ns or less, or the like) to receive sub-mV echoes from imaging targets less than 5 mm from the transducer, less than 3 mm from the transducer, less than 2 mm from the transducer, less than 1 mm from the transducer, less than 0.5 mm from the transducer, or the like.

The controller 20 and/or the power source 28 can be in wired communication with the interface chip 12 via the micro-cable(s) 18. The controller 20 can include a non-transitory memory (e.g., memory 22) that can store instructions for at least performing IVUS imaging and a processor 24 that can execute the instruction for at least performing IVUS imaging. It should be noted that the memory 22 and processor 24 can be separate devices or can be together as one device (e.g., a microprocessor). The power source 28 can be a portable power source and/or a non-portable power source such as a standard outlet connection to a power grid. The power source 28 can supply power to the IVUS interface chip 12 and the transducer 12 and may also supply power to the controller 20 and/or the display and user interface 26. The controller 20 can send instructions for IVUS signaling for pulsing mode of the dual-stage amplifier and can receive signals indicative of the echoes received during echo reception mode. The controller 20 can then analyze and configure the echoes using common ultrasound techniques to form a visualization of the imaging target on the display of the display and user interface 26. The display and user interface 26 can provide the visualization of the imaging target to at least one user (e.g., to at least one medical professional, patient, or the like), allow the at least one user to choose and/or modify IVUS parameters, control the movement of the IVUS catheter, and/or interact with the visualization. The display and user interface 26 can be, for example, a computer a smart phone, a tablet, an extended reality (XR) system (e.g., augmented reality, virtual reality, and/or mixed reality), a headset, or the like with a mouse, keyboard, touch screen, one or more buttons, motion capture interface, brain interface, or audio (microphone) interface, or the like. From the visualization the at least on user can determine and provide diagnostic, intervention and/or treatment next steps.

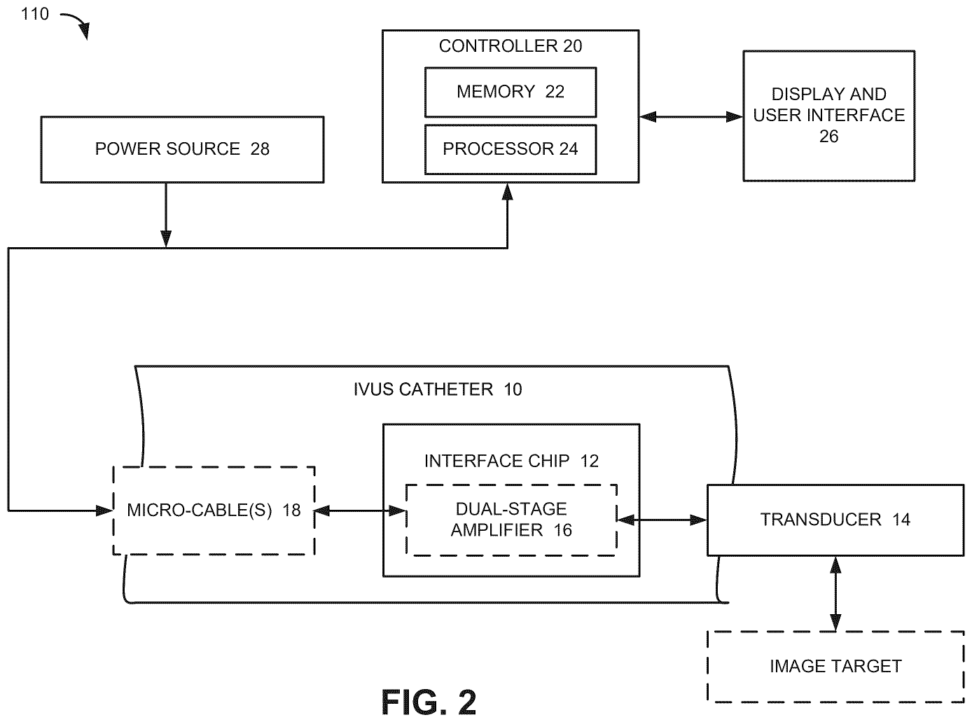

FIG. 3 shows several example configurations of IVUS systems (e.g., systems 120, 130, 140, and 150). It should be noted that any components not shown in FIG. 3, but shown in other FIGS., are omitted for ease of illustration only and should be understood that one or more of the components may be present. FIG. 3, element A and FIG. 3, element C show block diagram examples of at least partial IVUS imaging systems and FIG. 3, element B and FIG. 3, element D show electrical diagram examples of at least partial IVUS imaging systems. FIG. 3, element A and FIG. 3, element B correspond and FIG. 3, element C and FIG. 3, element D correspond. The micro-cables 1 and 2 18(1) and 18(2) can connect the transducer 14 and the interface chip 12 with a controller 20 and a power source (not shown). The micro-cables 1 and 2 18(1) and 18(2) (also referred to as micro-coax) can have a length of one meter or more (e.g., between 1 m to 2 m, between 1 m and 3 m, between 1 m and 5 m, or the like). The shields of the micro-cables 1 and 2 18(1) and 18(2) can be shared for power and ground and the interior can carry the voltage pulses to the transduce rand the echo output to the controller.

FIG. 3, element A shows a system 120 for IVUS imaging that can include a controller 20 (and a power source, not shown for ease of illustration) for providing a pulse input to an interface chip 12 (e.g., an AFE-ASIC) via micro-cable 1 18(1) that extends through the IVUS catheter 10. An element or set of elements on the interface chip 12 can then amplify and provide the pulse input signal to the transducer 14 via the dual-stage amplifier (not shown for ease of illustration). The transducer 14 can emit the pulse to the image target. The transducer 14 is shown outside of the IVUS catheter 10 in this instance, but may be in, on, and/or otherwise attached to the IVUS catheter. The transducer 14 can receive the echo from the pulse interacting with the image target (e.g., at least partially reflecting, absorbing, transmitting and/or otherwise changing). The interface chip 12 with the dual-stage amplifier can recover pulsing mode and move into echo receiving mode within the time the echo takes to reach the transducer 14. The interface chip 12 can send the echo to the controller 20 via the micro-cable 2 18(2). This process can be repeated for any number of pulses and echoes. The controller 20 can receive and configure the one or more echoes into a visualization of the image target for diagnosis, intervention, and/or treatment determination by a medical professional. FIG. 3, element B shows system 130, which is an electrical diagram version of the system 120 of FIG. 3, element A. It should be noted that the dual-stage amplifier on the interface chip 12 is configured to only need four conductors that include the two micro-cables (e.g., micro-coax) as connections. The shields of the micro-coaxes can be shared for power and ground, instead of just used for ground, which is the traditional method, while the interior of the micro-coaxes carry the pulses and the received and buffered echoes. The number of wires is a limiting factor in IVUS catheter size (increasing the number of wires increases the size required for the IVUS catheter 10 and the interface chip 12), so the fewer wires the better.

FIG. 3, element C shows a system 140 for IVUS imaging that can include a controller 20 (and a power source, not shown for ease of illustration) for providing a pulse input to an interface chip 12 (e.g., an AFE-ASIC) via micro-cable 1 18(1) that extends through the IVUS catheter 10. The input from the controller 20 (and/or power source) can pass through an AC blocking choke 21 before entering the micro-cable 1 18(1). The AC blocking choke 21 (which can be an inductor, for example) can block high-frequency alternating current and lower direct current and lower frequency alternating current from passing through. An AC blocking choke 21 can separate signals of different frequencies and prevent electromagnetic and/or radiofrequency interference. Another AC blocking choke can be positioned to between at least a portion of the micro-cable 1 18(1) and the interface chip 12 to receive and alter at least a portion of the pulse input. The interface chip 12 can then amplify and provide the pulse input signal to the transducer 14 via the dual-stage amplifier (not shown for ease of illustration). The transducer 14 can emit the pulse to the image target. The transducer 14 is shown outside of the IVUS catheter 10 in this instance, but may be in, on, and/or otherwise attached to the IVUS catheter. The transducer 14 can receive the echo from the pulse interacting with the image target (e.g., at least partially reflecting, absorbing, transmitting and/or otherwise changing). The interface chip 12 with the dual-stage amplifier can recover pulsing mode and move into echo receiving mode within the time the echo takes to reach the transducer 14. The interface chip 12 can send the echo to the controller 20 via the micro-cable 2 18(2). This can repeat for any number of pulses and echoes. The controller 20 can receive and configure the one or more echoes into a visualization of the image target for diagnosis, intervention, and/or treatment determination by a medical professional. FIG. 3, element D shows system 150, which is an electrical diagram version of the system 140 of FIG. 3, element B. It should be noted that in this configuration both shields can be connected together, formed with a triaxial cable, or formed as a twisted pair with shield (which would include only 3 conductors).

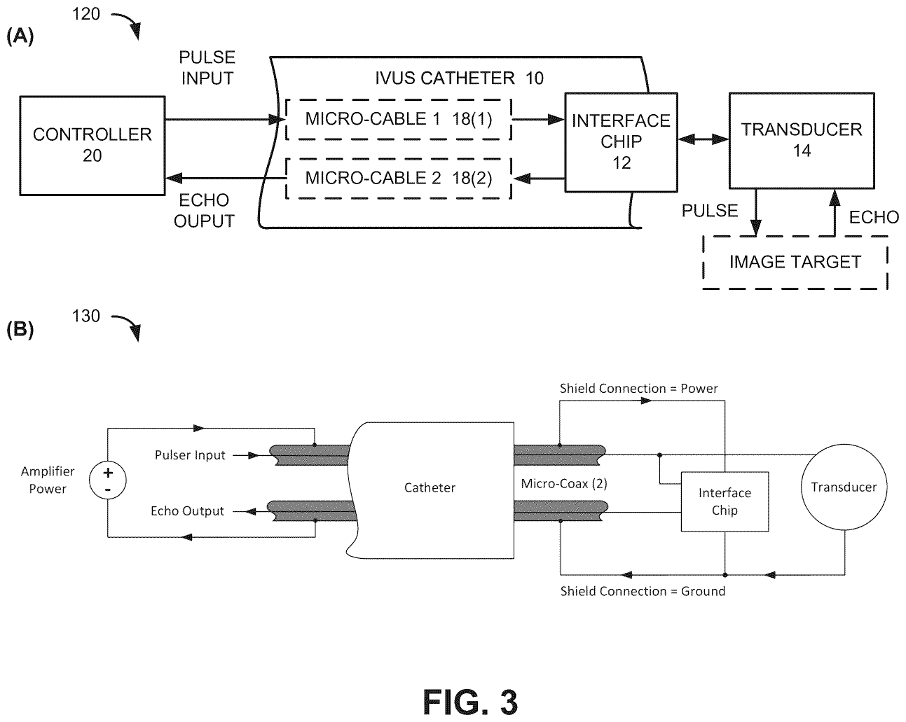

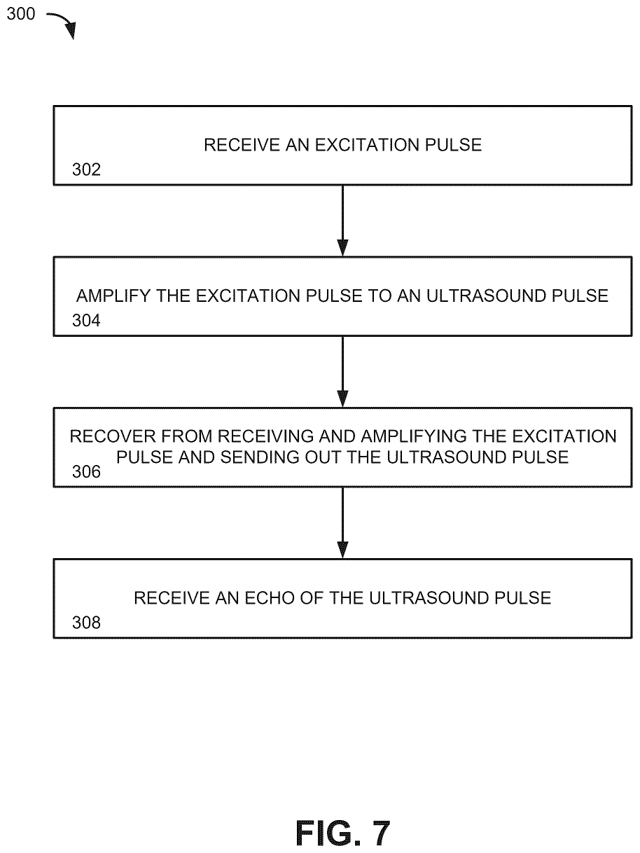

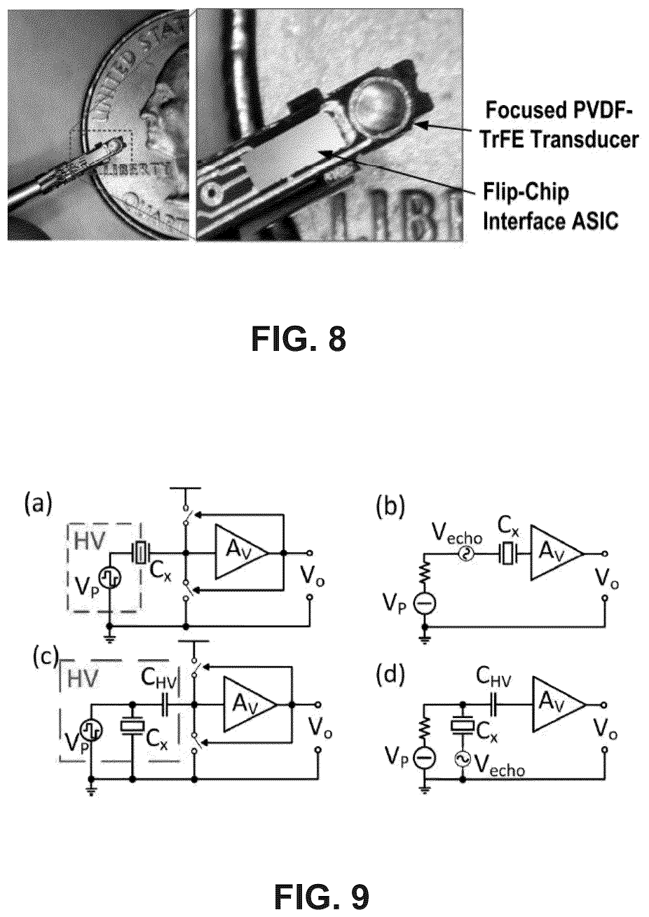

FIG. 4 shows the dual-stage amplifier 16 (which can be on the interface chip 12) in greater detail. For example, the dual-stage amplifier 16 on the interface chip 12 can be an analog front end (AFE) application specific integrated circuit (ASIC). As previously noted, the interface chip 12 can be sized to fit within the tip of a catheter (e.g., having a diameter of 2 mm or less, 1 mm or less, 0.5 mm or less, etc., but may be larger). The dual-stage amplifier 16 can be sized to fit on the interface chip and within the tip of the catheter. The dual-stage amplifier can include a transimpedance amplifier 30, an active limiter 32, and a buffer 34. The transimpedance amplifier 30 can be a feedback resistor (R_{F}) across an operational amplifier circuit that can convert current to a voltage drop using Ohm's law (V_{OUT}=i*R_{F}). The active limiter 32 can switch the dual-stage amplifier 16 from pulsing mode to echo reception mode quickly (e.g., within the time it takes to emit the pulse and receive the echo from imaging targets less than 5 mm from the transducer, less than 3 mm from the transducer, less than 2 mm from the transducer, less than 1 mm from the transducer, less than 0.5 mm from the transducer, or the like. For instance, quickly can be 1000 ns or less, 750 ns or less, 500 ns or less, 300 ns or less, or the like. The active limiter 32 can be an active current limiter, and may include a regenerative limiter 38, which employs positive feedback to amplify a signal, and/or a clamping limiter 40, which limits the output voltage to a specific range. In some instances, the active limiter 32 can also include an off-chip high voltage coupling capacitor 36. The off-chip high voltage coupling capacitor 36 can protect the integrated circuit (of the interface chip 12) from damage due to the voltage pulses that would damage the chip (e.g., pulses greater than 30 V_{PP}) during pulsing and can demonstrate low distortion during echo reception. When an off-chip high voltage coupling capacitor 36 is present, then the dual-stage-amplifier 16 and the off-chip high voltage coupling capacitor can be in a shunt duplexer connection to the transducer 14 (e.g., the ultrasound transducer). In another instance the dual-stage amplifier 16 can be in a series duplexer connection to the transducer 14 (e.g., the ultrasound transducer) (e.g., without using an off-chip high voltage coupling capacitor 36). The buffer 34 can preserve the bandwidth of the transducer over the length of the micro-cable (e.g., micro-cable 18(2)) and can buffer the echo input received from the transducer 14 during echo reception. For example, the buffer 34 can be at least a 50-ohm buffer. The active limiter 32 can be activated by feedback from the transimpedance amplifier (e.g., sending the pulse) or the buffer (e.g., receiving the echo). In some instances, the transimpedance amplifier 30 can take the place of the buffer 34 if the gain is large enough to handle the cable driving/buffering function. Example circuits can be seen in FIGS. 9 and 10, discussed in greater detail below.

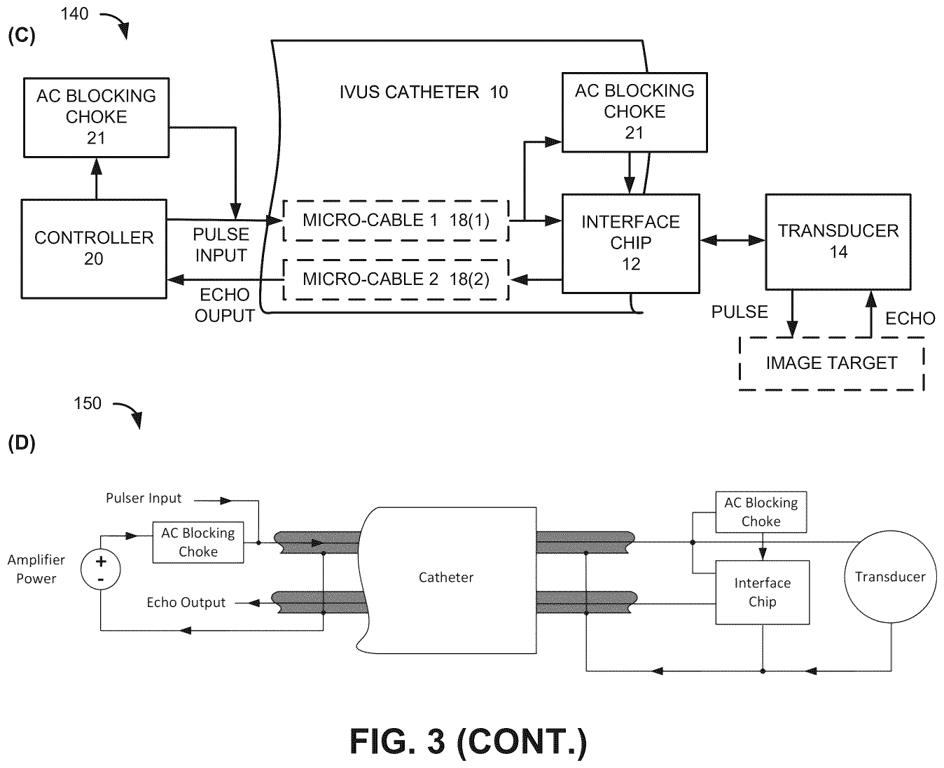

FIG. 5 shows a system 160 that can include at least the dual-stage amplifier 16 and the transducer 14. The dual-stage amplifier 16 can be a custom analog front end (AFE) ASIC. The dual-stage amplifier 16 can connect the transducer 14 (e.g., an ultrasound transducer) of an intravascular ultrasound to a controller (not shown in this figure for ease of illustration, but e.g., controller 20 via at least two micro-cables, but may also connect to a power source or another component). The connection between the transducer 14 and the dual-stage amplifier 16 can allow bi-directional communication and can be a shunt duplexer connection 42 or a series duplexer connection 44.

The dual-stage amplifier 16 can be shaped and sized to fit within the tip of an intravascular ultrasound (IVUS) catheter having a diameter of 2 mm or less, 1 mm or less, 0.5 mm or less, or the like (in some instances, the diameter may be larger). Similar to what is described in FIG. 4, the dual-stage amplifier can include a transimpedance amplifier 30, an active limiter 32, and a buffer 34. The transimpedance amplifier 30 is an operational amplifier with a feedback resistor that can convert current to voltage. The active limiter 32 can switch the dual-stage amplifier 16 from pulsing mode to echo reception mode quickly (e.g., within the time it takes to emit the pulse and receive the echo from imaging targets less than 5 mm from the transducer, less than 4 mm from the transducer, less than 3 mm from the transducer, less than 2 mm from the transducer, less than 1 mm from the transducer, less than 0.5 mm from the transducer, or the like. For instance, quickly can be within 1000 ns or less, 750 ns or less, 500 ns or less, 300 ns or less, or the like. The active limiter 32 can be configured for symmetrical switching, where the active limiter can be activated by hysteresis enabled positive feedback from the buffer 34 and/or the transimpedance amplifier 30. The active limiter 32 can protect the dual-stage amplifier 16 from damage due to excitation pulse inputs that would damage the chip (e.g., pulses greater than 30 V_{PP}) during pulsing and can provide low distortion during echo reception.

In some instances, the active limiter 32 can also include an off-chip high voltage coupling capacitor 36. When an off-chip high voltage coupling capacitor 36 is present, then the dual-stage amplifier 16 and the off-chip high voltage coupling capacitor can be in a shunt duplexer connection to the transducer 14 (e.g., the ultrasound transducer). In another instance the dual-stage amplifier 16 via at least the active limiter 32 can be in a series duplexer connection to the transducer 14 (e.g., the ultrasound transducer) (e.g., without using an off-chip high voltage coupling capacitor 36). The buffer 34 can preserve the bandwidth of the transducer over the length of the micro-cable (e.g., micro-cable 18(2)) and can buffer the echo input received from the transducer 14 during echo reception. The buffer 34 can be at least a 50-ohm buffer. The active limiter 32 can be activated by feedback from the transimpedance amplifier (e.g., sending the pulse) or the buffer (e.g., receiving the echo). In some instance, the transimpedance amplifier 30 can take the place of the buffer 34 if the gain is great enough to handle the cable driving/buffering function.

### IV. Methods

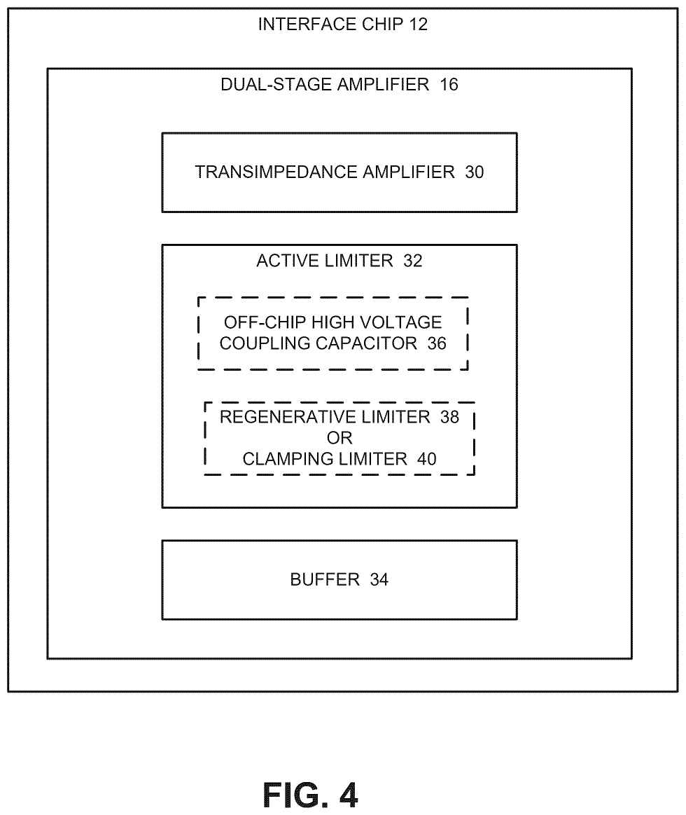

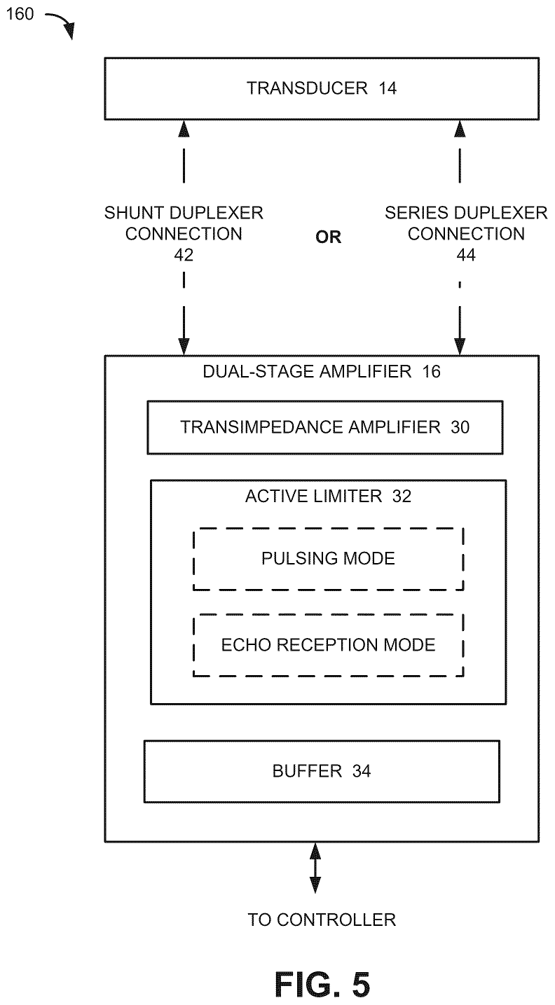

Another aspect of the present disclosure can include methods 200 (FIG. 6) and 300 (FIG. 7) for intravascular ultrasound of an artery and/or vein within the peripheral vasculature. The methods 200 and 300 can be executed using at least one of the system 100, 110, 120, 130, 140, 150, and/or 160 shown in FIGS. 1, 2, 3, and 5. For purposes of simplicity, the methods 200 and 300 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 200 and 300, nor are methods 200 and 300 limited to the illustrated aspects.

FIG. 6 shows a method 200 for performing intravascular ultrasound (IVUS) imaging in peripheral vasculature. For example, for a vascular intervention (e.g., diagnostic, treatment, removal, etc.) in a peripheral artery or vein. IVUS offers a high spatial resolution and enables real-time investigation before, during, or following a procedure. Peripheral arteries and/or veins require smaller catheters with broader transducer bandwidths to achieve the necessary microscopic resolution needed to provide useful images (e.g., for the detection of legions, other pathologies, stent pieces or location, or the like). To enable such broad bandwidths a sub-millimeter IVUS catheter (e.g., catheter 10) can include an interface chip (e.g., interface chip 12) having a dual-stage amplifier (e.g., dual-stage amplifier 16) connected to a transducer (e.g., transducer 14, which can be a PVDF-TrFE transducer) that can amplify the local sensor signal (e.g., sub-mV), withstand pulses that would otherwise damage the dual-stage amplifier or chip (e.g. greater than 100 V_{PP}) and recover quickly enough (e.g., within 1000 nanoseconds or less) to receive sub-mV echoes from targets less than 3 mm (or smaller, such as less than 1 mm, less than 0.5 mm, or the like) from the transducer. The interface chip can be configured to have an improved signal-to-noise ratio (SNR), a rapid clamping limiter, and supports both series- and shunt-mode duplexing with the transducer (as discussed above in the System and below in the Experimental).

At 202, the IVUS catheter (e.g., IVUS catheter 10) can be introduced into a peripheral artery or vein of interest. For example, the IVUS catheter can be 3 French or less (e.g., diameter of 1 mm or less) to fit within the small peripheral arteries and/or veins. The interface chip (e.g., interface chip 12) can be integrated into the tip of the IVUS catheter and connected between the transducer (e.g., transducer 14) and the power (e.g., power source 28) and control (e.g., controller 20) portions of the IVUS system. At least two micro-cables (e.g., micro-cables 18) having a shield communicating both power and ground can connect the interface chip to the power and control portions of the IVUS system. A medical professional can determine which artery or vein should be imaged and can manually, semi-manually, or automatically (e.g., with the assistance of a surgical robot or the like) introduce the IVUS catheter into the artery or vein (e.g., through a surgical opening). It should be noted that this procedure can be repeated for each artery and/or vein of interest.

At 204, an ultrasound pulse can be emitted via the transducer at a given frequency. For example, at a high frequency (e.g., 10 MHz or greater, 20 MHz or greater, 30 MHz or greater, 40 MHz or greater, or the like) depending on the size of the vein or artery and/or the suspected pathology or imaging target. The frequency can be determined via a controller in electrical communication with the transducer via at least the interface chip and the at least two micro-cable within the IVUS catheter. The interface chip, including the dual-stage amplifier, can amplify the signal of the ultrasound pulse prior to it being emitted by the transducer. At 206, an echo can be received via the transducer. The echo can be an echo of the ultrasound pulse reflecting off at least one imaging target such as a pathology (e.g., a lesion), the arterial and/or venous tissue, blood, a stent or other medical device, or the like. The characteristics of the echo can change based on the composition of the imaging target the ultrasound pulse reflected off of and how far away from the transducer it was. Between sending out the ultrasound pulse and receiving the echo, the interface chip including the dual-stage amplifier undergoes pulse recovery. For instance, pulse recovery to greater than 230 V_{PP} can occur, for instance, between 325 ns and 450 ns, which allows for imaging as close as 0.5 mm from the transducer. Pulse emission and echo reception can be repeated any number of times (e.g., as the IVUS catheter is translated and/or rotated within the vein and/or artery) to form an entire visualization. Steps 204 and 206 can be repeated.

At 208 an IVUS image of at least the peripheral artery or vein (and any other components and/or pathologies therein) can be formed (e.g., visualized on a display) at a microscopic resolution. The image can be formed on a display associated with (e.g., in wired and/or wireless electrical communication) the controller of the IVUS system based on the received echoes. For instance, images can be obtained from a 0.88 mm, 40 Hz transducer with sufficient resolution to detect individual stent struts within an artery having a wall thickness of 0.35 mm. The images can be used for diagnosis, treatment, concurrent intervention within the artery or vein, or the like.

FIG. 7 shows a method 300 for use of the interface chip (e.g., interface chip 12) including the dual-stage amplifier (e.g., dual-stage amplifier 16) for IVUS imaging. An excitation pulse can be sent to a dual-stage amplifier on an interface chip via a micro-cable including both power and ground in a shield. At 302, the dual-stage-amplifier can receive the excitation pulse. A transimpedance amplifier within the dual-stage amplifier can convert the current to a voltage output. At 304 the excitation pulse can be amplified to an ultrasound pulse (e.g., via an active limiter) and sent to the transducer for emission. Once the ultrasound pulse is emitted it can echo off an imaging target (e.g., the pulse can be reflected, absorbed, transmitted, or otherwise changed depending on the composition of the image target, as is known in the art). At 306, while the ultrasound pulse is being emitted by the transducer, traveling through the body, and echoing off the image target, the dual-stage amplifier of the interface chip can recover from receiving and amplifying the excitation pulse and sending out the ultrasound pulse. The recovery time can be less than 1000 ns, less than 500 ns, less than 300 ns or the like, to allow recovery before an echo can be received from an image target 3 mm or less, 1 mm or less, or 0.5 mm or less from the transducer. At 308, an echo from the ultrasound pulse can be received by the recovered dual-stage amplifier and then stent to the controller (e.g., via the micro-cable 2 18(2)). The echo may be buffered by a buffer of the dual-stage amplifier before entering the micro-cable. This can repeat for any number of pulses and echoes. The controller can the receive and configure the one or more echoes into a visualization of the image target for diagnosis, intervention, and/or treatment determination by a medical professional.

### V. Experimental

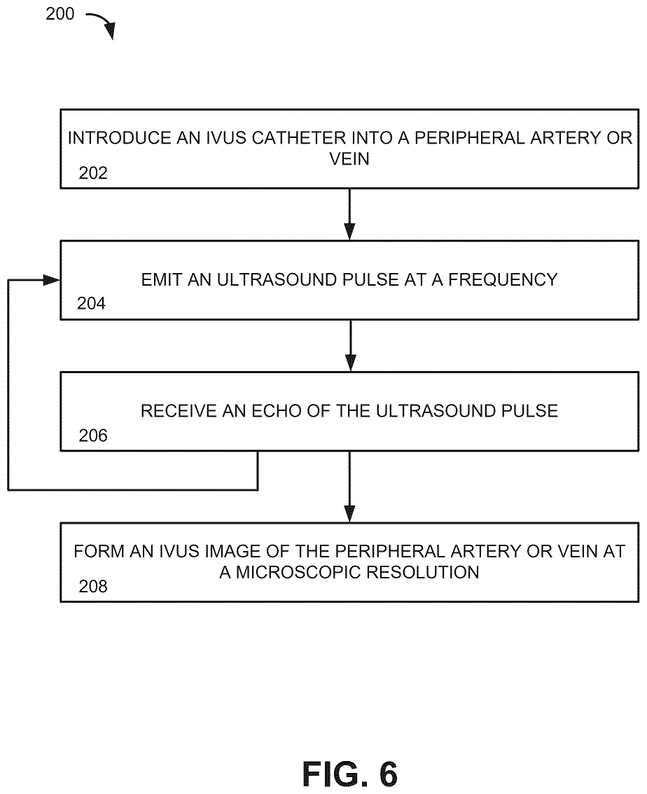

The following experiment presents a dual-channel interface analog front end (AFE) application specific integrated circuit (ASIC) for local sensor signal amplification in a poly[(vinylidenefluoride-co-trifluoroethylene](PVDF-TrFE) IVUS. Practical application of PVDF-TrFE IVUS requires local sensor signal amplification due to the high transducer impedance and low echo reception level. The AFE ASIC was developed to withstand pulses greater than 100 V_{PP} while recovering quickly enough to receive sub-mV echoes from targets less than 0.5 mm from the transducer. FIG. 8 shows an example depicting an AFE ASIC and broadband PVDF-TrFE transducer in a size suitable for IVUS imaging of coronary arteries (on the left, the AFE ASCIC and broadband PVDF-TrFE transducer is much smaller than a quarter). The ASIC dimensions were less than 0.5 mm in width to enable flip-chip integration at the tip of an IVUS catheter less than 3 French (1 mm diameter) (see FIG. 8).

### I. High-Impedance Analog Front-End

### A. Pulse-Limiting Strategy

Recent examples of CMUT ultrasonic systems, including those used for IVUS, and arrays for cardiac imaging, integrate on-chip pulser circuitry and analog front-ends (AFEs) for echo reception. Due to the lower piezoelectric coefficient of PVDF-TrFE PMUTs, large voltages are needed to excite the transducer. Because modern integrated circuit processes do not include high-voltage FETs capable of withstanding 50-100 V, a shunt/series-duplexer topology was used rather than a traditional AFE with a transmit/receive switch (see FIG. 9). The AFE was designed to support both duplexer topologies using an optional external high-voltage coupling capacitor CHV. Please note this was for experimental purposes and an AFE may be configured with only a shunt or a series duplexer topology. FIG. 9 shows equivalent duplexer circuits during pulsing and echo reception modes. FIG. 9, element (a) shows the shunt duplexer clamping pulse. FIG. 9, element (b) shows the shunt duplexer receiving echo assuming the pulser V_{P} remains low impedance. FIG. 9, element (c) shows series duplexer clamping pulse with limited loading by C_{HV}. FIG. 9, element (d) shows series duplexer echo reception demonstrating C_{HV} and V_{P} insertion loss.

During pulsing, the active limiter clamps the input voltage to less than 3.3V to prevent damage to the AFE input transistors (see FIG. 9). The FETs in the active limiter were sized to clamp current during pulsing; current was also limited by the value of the off-chip capacitor C_{HV}. The size of C_{HV}, AFE input capacitance, and limiter FETs size were calculated using an iterative optimization process to maximize SNR, as described previously. Due to the small capacitance of the polymer PMUT and the inherent input capacitance of the AFE, the shunt duplexer topology led to an expected insertion loss of 12 dB (see FIG. 9), even after optimization.

### B. Buffer Transimpedance Amplifier

FIG. 10 shows proposed AFE ASIC schematics. FIG. 10, element(a) shows the proposed AFE ASIC schematic connected with the external IVUS pulse system through an IVUS catheter. The AFE ASIC consists of an HV coupling capacitor C_{HV}, transimpedance amplifier, active pulse limiter, and a 50Ω buffer integrated at the catheter tip to preserve the wide transducer bandwidth over a long cable. C_{HV} is an off-chip R_{F} capacitor that must withstand 250 V during pulsing and demonstrate low distortion during echo reception.

FIG. 10, element (b) and element (c) show the AFE ASIC including two amplifier channels (CH1 and CH2, respectively) which used different limiters for active clamping. CH2 additionally included a bondpad output between the low-noise amplifier (LNA) stage and output buffer; parasitic loading slightly reduced CH2 bandwidth as a result. FIG. 10, element (b), shows AFE CH1 using a clamping limiter which activates when amplifier V_{IN} and V_{OUT} differ by more than a MOS VT. As shown in FIG. 10, element (b) AFE CH1 used a complimentary common-source transimpedance LNA (M₁, M₂). FIG. 10, element (c) shows AFE CH2 using a regenerative limiter for faster clamp activation, and a swing-limited LNA to aid in bias point overload recovery. As shown in FIG. 10, element (c) CH2 used the same LNA (as CH1) with a swing limiting feedback network (M₁, M₂). The swing-limiter was used to prevent overload and speed recovery after ultrasound pulsing. The network effectively limits the CH2 swing to +/−one MOS VT, and channel length devices were used to minimize distortion at lower amplitudes.

The LNA amplifies and sets the bandwidth of the echo signal. A low overall gain and a single stage amplifier was used to maintain linearity and prevent harmonic generation; harmonic distortion would prevent accurate multi-frequency or harmonic imaging. The feedback resistance R_{f} was designed to set the amplifier gain with an assumed transducer capacitance of 2 pF. The parasitic capacitor C_{f} is formed due to overlap capacitance in the MOS structure and limits the AFE bandwidth due to Miller feedback. In this application, however, only 100 MHz bandwidth was needed, so R_{f} was set to 380 kΩ.

### C. Regenerative and Linear Active Limiter Structures

To protect the amplifier from the high pulsing voltage, the active limiter (M_{LIM1}, M_{LIM2}) is implemented to ensure that the voltage during the pulsing period remains below the FET gate breakdown voltage. During high-voltage pulsing, feedback buffers monitored the output voltage of the amplifier (see FIG. 10, element (b)) and activated the limiter switches accordingly (see FIG. 10, element (a)). Limiter switches were turned on proportionally via the LNA output rather than hard clamping to the supply rails by feeding back the inverted LNA output to the limiter gates during clamping.

This feedback effectively restricted the swing of the LNA output voltage to +/−approximately one MOS VT, providing reliable voltage clamping and preventing any potential damage to the FET gate. AFE CH2 (see FIG. 10, element (c)) used the improved regenerative limiter for faster clamp activation, which will lead to a faster recovery during the pulse phase. Unlike the CH1 limiter, the regenerative limiter included a cross-coupled pair for hysteretic positive feedback to accelerate switching faster than the LNA slew rate on the limiter gate capacitance.

### II. Proposed AFE ASIC Characterization

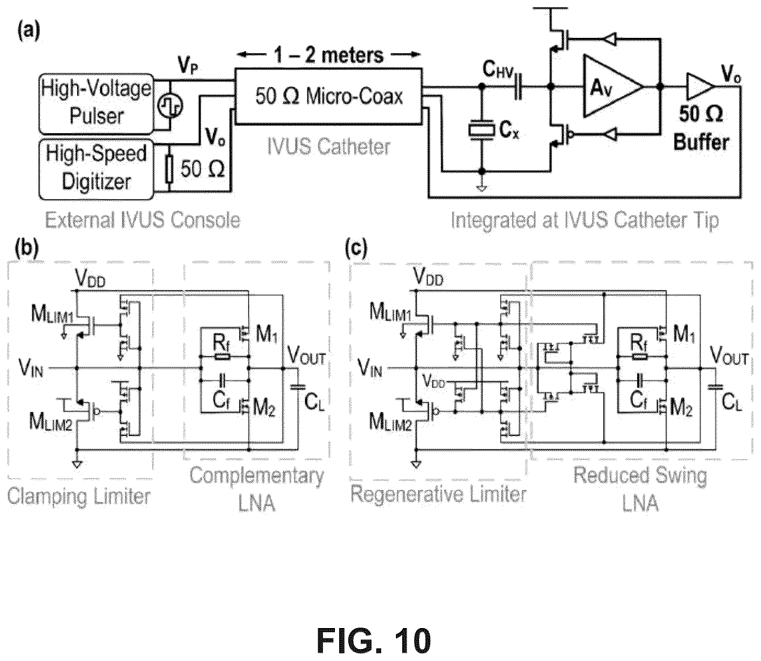

The AFE ASIC was fabricated in the TSMC RF/LP 65 nm process. FIG. 11 shows close up views of an example fabricated AFE test chip. FIG. 11, element (a) shows the AFE test chip was 0.5 mm wide to enable a sub-mm IVUS catheter. Each die (including test circuits) measured 1.00 mm×0.5 mm; so each AFE occupied 0.032 mm² die area (core area) with the remaining space used for text circuits. As shown in FIG. 11, element (b) and element (c) AFE circuits (chips) were wire bonded into QFN24 packages (see FIG. 11, element (b)) for bench characterization (see FIG. 11, element(c)).

### A. Amplifier Analog Performance Characterization

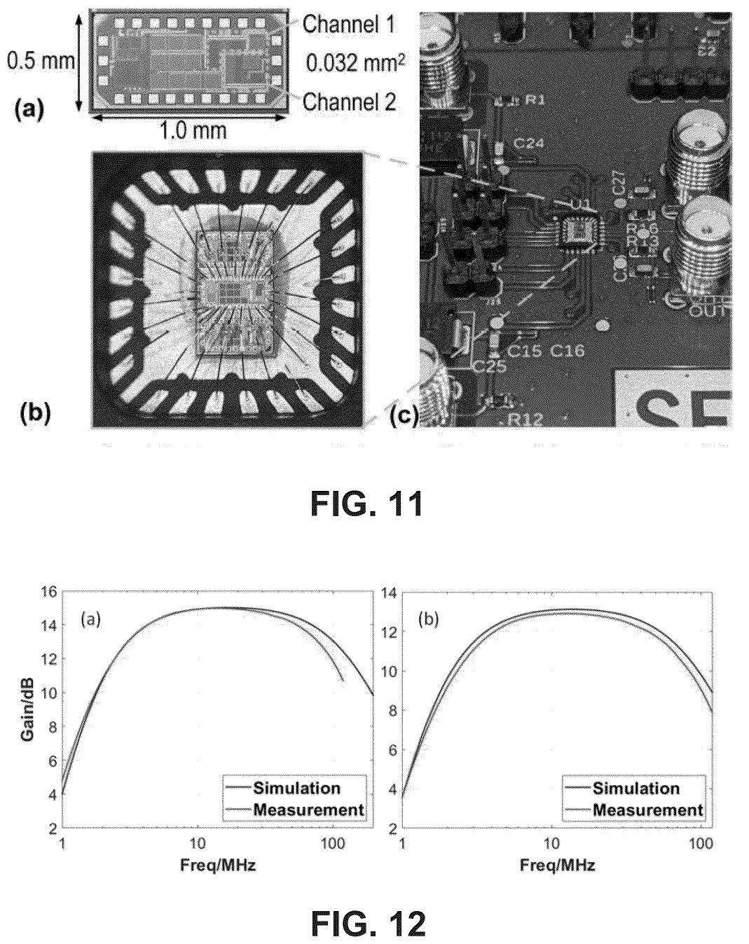

FIG. 12 shows a graphical representation of the S21 measurement and simulation results. FIG. 12, element (a) shows results for CH1 and FIG. 12, element b shows results for CH2. Noise and gain parameters of the AFE signal chain were tested using a discrete 2 pF capacitor to represent the PMUT (Keysight ENA Network Analyzer E5061 B). All measured results used a 50-0 input and load (Table 1). The reported SNR assumed a 1-mV echo and the full amplifier 3 dB bandwidth with no spectral leakage. The low thermal noise (2.1 nV/NHz) of both CH1 and CH2 would allow for much higher SNR within a fundamental-only 40 MHz bandwidth, at the expense of image resolution. CH2 bandwidth was limited by parasitic capacitance from the inclusion of a test bondpad for characterizing the 50-Ω driver. Mismatch loss from the driver was minimal at 0.3 dB of lost signal when driving a 50-Ω coax and termination.

### B. Pulse Recovery

IVUS systems work by transmitting an electrically actuated acoustic pulse and receiving an echo shortly after; the timing between pulse and echo is determined by the speed of sound in blood and the distance to the target. Because IVUS is used in small-caliber vessels the AFE ASIC was designed to receive and echo within 667 ns of pulsing (0.5 mm echo distance). Pulse recovery testing used a high-voltage pulser (Avtech AVB1-3-C, Avtech Electrosystems) to apply a 40 MHz monocycle pulse to the AFE input. This test demonstrated both the active limiter's clamping ability and the AFE's post-pulse recovery time. These tests were vital in ensuring that the amplifier could swiftly resume operation and effectively capture the subsequent echo signals for imaging. FIG. 13 shows graphical representations of recovery after limiting high-voltage pulse, FIG. 13, element (a) shows CH1 and FIG. 13, element (b) shows CH2. FIG. 14 shows graphical representations of the pulse amplitude vs recovery time, with FIG. 14, element (a) showing CH1 and FIG. 14, element (b) showing CH2. As shown in FIG. 13, when a 100V pulse voltage was applied to the input, the AFE's output voltage was kept well below the breakdown voltage, and the amplifier of CH1 recovered ˜450 ns after pulsing. CH2, due to the fast clamping design, had a better recovery time of ˜325 ns (see FIG. 14). Results illustrated that the proposed AFE ASIC CH1/CH2 handled peak pulse voltages up to 115 V/125 V with recovery times less than 600 ns/450 ns.

TABLE I
Summary of AFE Measured Performance

| Measured Parameter | CH1 | | CH2 | |
|---|---|---|---|---|
| Loaded AFE Gain (40 MHz, including | 14.2 | dB | 12.7 | dB |
| transducer insertion loss) | | | | |
| Bandwidth (−3 dB) | 2-107 | MHz | 2-86 | MHz |
| Thermal Noise Floor | 2.1 | nV/√Hz | 2.1 | nV/√Hz |
| Integrated Transducer-Referred Noise | 21.6 | μV_{RMS} | 21.4 | μV_{RMS} |
| Mismatch Loss at 40 MHz (50-Ω load) | 0.3 | dB | 0.3 | dB |
| Full-Band Signal to Noise Ratio | 33.3 | dB | 33.4 | dB |
| Pulse Voltage Handling | >230 | V_{PP} | >250 | V_{PP} |
| 200 V_{PP} Pulse Recovery Time | 450 | ns | 325 | ns |

*Assuming 1 mV_{RMS} acoustic echo received by PMUT during imaging

### III. Example Broad-Band IVUS Image

### A. Polymer Micro-Focused Ultrasonic Transducer

A 0.8-mm aperture PVDF-TrFE PMUT was fabricated to demonstrate AFE imaging performance. The PMUT was produced as previously described and embedded directly within a Rogers 4350B PCB substrate. Briefly, the PMUT was formed by pressing a spherical focusing die to a 9 μm film of Chrome Gold-metallized PVDF-TrFE. The film thickness determined the primary resonance frequency of 40 MHz for the transducer. The film was held in place and backed by an electrically conductive epoxy which also contacted the active transducer face. Conductive epoxy was applied via syringe dispensing to a contact on a test PCB. The aperture size (0.8 mm) was controlled by a drilled PCB hold, while the focal length (3 mm) was set by the radius of the spherical forming tool. The 0.8-mm transducer was modeled as a 2-pF capacitor based on previous characterization.

### B. PCB Tower for Bench Imaging Demonstration

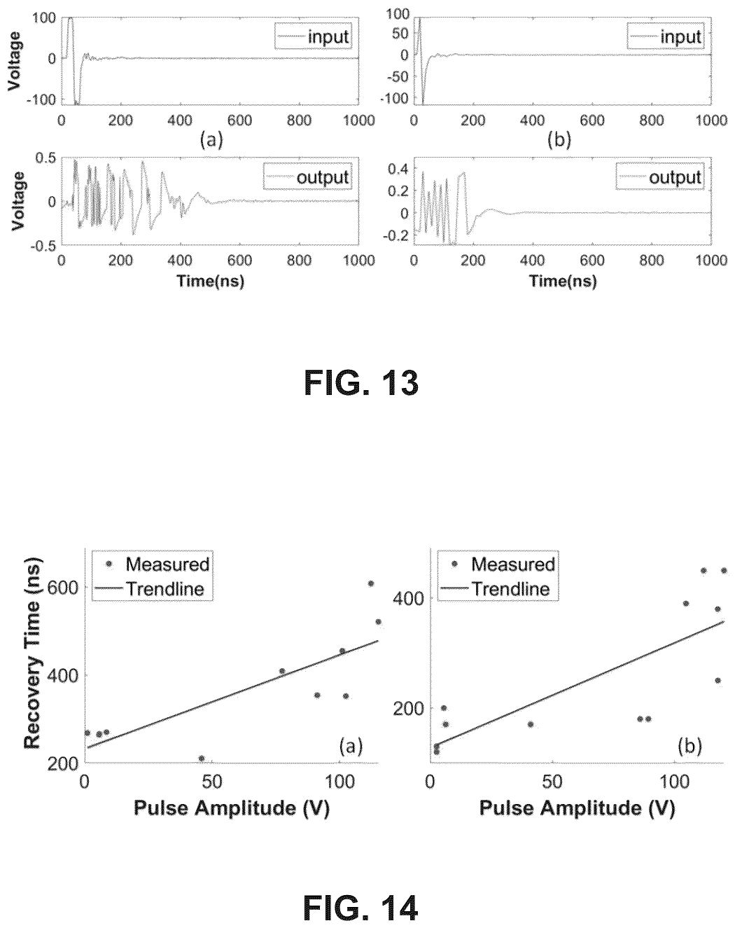

A specially designed tower-shaped PCB, shown in FIG. 15, element (a), was created to allow basic imaging with the AFE ASIC and the polymer PMUT in a form factor larger than a catheter. The tower-shaped PCB was designed to house the AFE ASIC. The tower-shaped PCB tapered to a 1.5-mm diameter tip, which housed the 0.8-mm PMUT embedded at the narrow tip. This test used a previous iteration of the AFE described here, with similar bandwidth but worse SNR. While the presented AFE is expected to provide improved image quality, imaging experiments were performed to demonstrate the active limiter topology did not introduce distortion or other artifacts to the IVUS image.

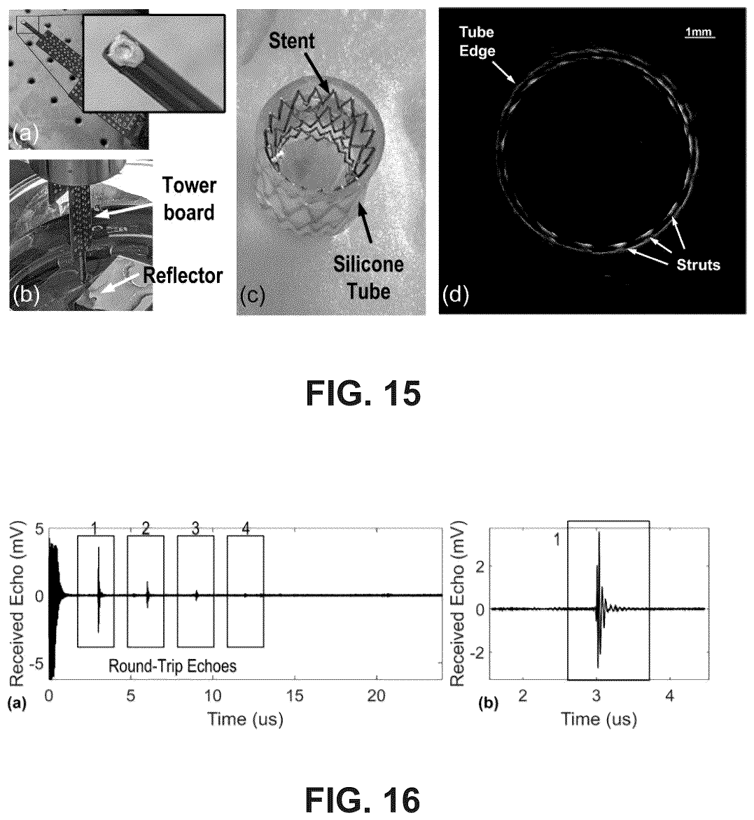

The AFE ASIC was connected to a commercial IVUS pulser and a sampling oscilloscope using a 50Ω coaxial cable. To determine the focal length of the transducer, a pulse-echo test was conducted using a polished steel reflector immersed in deionized water. An example set up of the test is shown in FIG. 15, element (b). FIG. 16, elements (a) and (b) show graphical representations demonstrating the detection of four round-trip echoes from a single excitation pulse. The first echo exhibited an excellent signal-to-noise ratio (SNR) and achieved an approximately 98% 10-db fractional bandwidth. FIG. 16, element (a) shows a non-averaged A-line received from a polished steel reflector in DI water demonstrated detection of 4 round-trip echoes from a single excitation pulse. FIG. 16, element (b) shows the first reflected pulse having an excellent signal to noise ratio (SNR) and approximately 98% 10-db fractional bandwidth.

### C. Stepped Rotational Image Acquisition

Referring back to FIG. 15, FIG. 15 element (c) shows a phantom with a stent within a 6.7 mm outer diameter silicone tube used for testing. The stent phantom was produced for image demonstration by opening a 6-mm stent within a silicone tube and embedding the tube base in paraffin wax. The tube and surrounding area were filled with deionized water for imaging. The outer diameter of the silicone tube measured 6.7 mm, which placed it beyond the nominal focal range of 3 mm for the 0.8-mm transducer.

FIG. 15, element (d) shows a rotational IVUS image that was captured by inserting the tower-mounted transducer into a stent phantom and rotating the tower while recording pulse-echo waveforms (A-lines). One degree of rotation was used for waveform capture. Images were created from 360 A-lines with 5,120 points per line for a radial distance of 7.9 mm. Image dynamic range was scaled to 40 dB. Images were captured along the length of the stent and demonstrated clear detection of stent struts and the tube edges. The 40-MHz center frequency image demonstrated sufficient resolution for stent imaging, without shadowing behind stent struts which is an artifact of intra vascular optical coherence tomography (IVOCT).

From the above description, those skilled in the art will perceive improvements, changes, and modifications. Such improvements, changes and modifications are within the skill of one in the art and are intended to be covered by the appended claims.

The following is claimed:

1. A system comprising:
an intravascular ultrasound (IVUS) catheter;
an ultrasound transducer with a broad excitation bandwidth; and
an interface chip sized to be integrated within a tip of the IVUS catheter to connect micro-cables within the IVUS catheter to the ultrasound transducer and comprising a dual-stage amplifier having a pulsing mode configured to withstand voltage pulses greater than a voltage on a level of volts during pulsing and an echo reception mode configured to receive sub-mV echoes during echo reception.

2. The system of claim 1, wherein the dual-stage amplifier comprises:
a transimpedance amplifier;
an active limiter configured to switch between the pulsing mode and the echo reception mode quickly; and
a buffer.

3. The system of claim 2, wherein the dual-stage amplifier further comprises an off-chip high voltage coupling capacitor configured to protect the integrated circuit from damage due to the voltage pulses greater than the voltage on a level of 30 V_{PP} or more during pulsing and to demonstrate low distortion during the echo reception.

4. The system of claim 3, wherein the dual-stage amplifier and the off-chip high voltage coupling capacitor are in a shunt duplexer connection to the ultrasound transducer.

5. The system of claim 2, wherein the dual-stage amplifier has a series duplexer connection to the ultrasound transducer.

6. The system of claim 2, wherein the active limiter comprises a regenerative limiter or a clamping limiter.

7. The system of claim 2, wherein the buffer is at least a 50 Ohm buffer.

8. The system of claim 2, wherein the active limiter is activated by feedback from the transimpedance amplifier or the buffer.

9. The system of claim 8, wherein a swing of the output voltage is restricted to an absolute value of a threshold voltage for a MOSFET of the amplifier to provide voltage clamping while preventing damage to the MOSFET.

10. The system of claim 1, further comprising the micro-cables configured to connect the transducer and the interface chip with a power source and a controller, wherein the micro-cables have a length of one meter or more.

11. The system of claim 10, wherein the micro-cables are two micro-cables comprising shield connections that are shared for power and ground, while interiors of the micro-cables carry the voltage pulses to the transducer and an echo output to the controller.

12. The system of claim 1, wherein the dual-stage amplifier is an analog front end (AFE) application specific integrated circuit (ASIC) on the interface chip.

13. The system of claim 1, wherein the dual-stage amplifier is sized to fit within a diameter of the IVUS catheter of 1 mm or less.

14. The system of claim 1, wherein the dual-stage amplifier receives sub-mV echoes from targets less than 3 mm away from the ultrasound transducer.

15. A dual-stage amplifier that connects an ultrasound transducer of an intravascular ultrasound with a controller, the dual-stage amplifier comprising:
a transimpedance amplifier,
an active limiter comprising a pulsing channel configured to withstand voltage pulses greater than a voltage on a level of volts and an echo reception channel configured to receive sub-mV echoes; and
a buffer,
wherein the dual-stage amplifier is configured to fit within a tip of an intravascular ultrasound (IVUS) catheter.

16. The dual-stage amplifier of claim 15, wherein the active limiter is configured to protect the dual-stage amplifier from an excitation pulse input greater than 30 V_{PP} during pulsing and provide low distortion during echo reception.

17. The dual-stage amplifier of claim 15, wherein the active limiter is in shunt duplexer or a series-duplexer connection with the ultrasound transducer of an intravascular ultrasound (IVUS) system.

18. The dual-stage amplifier of claim 15, wherein the active limiter is in a shunt duplexer connection with the ultrasound transducer of an intravascular ultrasound (IVUS) system, the shunt duplexer connection further comprises an off-chip high voltage coupling capacitor.

19. The dual-stage amplifier of claim 15, wherein the active limiter is configured for symmetrical switching and wherein the active limiter is activated by hysteresis enabled positive feedback from the buffer and/or the transimpedance amplifier.

20. The dual-stage amplifier of claim 15, wherein the dual-stage amplifier is a custom analog front end (AFE) ASIC.
